(19) United States
(12) Patent Application Publication
(10) Pub. No.: US 2012/0071833 A1
(43) Pub. Date: Mar. 22, 2012

(54) IMPLANTABLE CATHETER OR LEAD ANCHOR FOR IMPLANTABLE MEDICAL DEVICE SYSTEM AND METHOD OF USE

(76) Inventors: Roger J. Hill, Richardson, TX (US); Brian Blischak, Allen, TX (US); Kurt Cantlon, Plano, TX (US)

(21) Appl. No.: 12/040,187

(22) Filed: Feb. 29, 2008

Related U.S. Application Data
(60) Provisional application No. 60/892,655, filed on Mar. 2, 2007.

(51) Int. Cl. A61M 25/04; A61N 1/05

(52) U.S. Cl. 604/175; 607/116

(57) ABSTRACT

In one embodiment, an implantable anchor for holding a catheter in place within a patient, comprises a body comprising a channel for holding the catheter; a plurality of apertures such that at least one aperture is disposed on each side of the spiral channel; and one or more suture structures to allow a surgeon to suture the anchor to tissue of the patient; wherein when a catheter is disposed within the channel and is threaded through the plurality of apertures, the plurality of apertures limit longitudinal displacement of the catheter in either longitudinal direction relative to the anchor without continuously applying a compressive force to the catheter.

### CROSS-REFERENCE TO RELATED APPLICATIONS

This application claims the benefit of U.S. Provisional Application No. 60/892,655, filed Mar. 2, 2007, the disclosure of which is incorporated herein by reference.

### BACKGROUND

The present application is generally related to an implantable anchor for anchoring a drug infusion catheter, an electrical stimulation lead, or other catheter of an implantable medical device system.

A number of implantable medical devices have been commercially distributed that allow various medical agents to be controllably infused after implantation of the respective device within a patient. For example, implantable medical devices are used for the infusion of insulin, opiates, anti-spasmodic drugs, intrahepatic chemotherapy agents, and other therapeutic agents in a number of countries subject to the regulatory requirements of those countries.

There are a number of benefits to the use of implantable infusion devices. For example, when the therapeutic agent is delivered directly to the therapy site (for opiates and baclofen), the amount of the therapeutic agent that is needed is much lower. Side-effects are generally minimized. Also, the therapeutic effect can be significantly greater as compared to intravenous introduction of therapeutic agents (again, for opiates and baclofen). Furthermore, implantable infusion devices eliminate patient overdosing or underdosing due to patient error or limited patient capacity.

Implantable infusion devices typically include a central housing that includes a reservoir to hold the infusate, a septum to allow infusate to be introduced into the reservoir, an energy source to drive the infusate from the reservoir and through an outlet port, and various flow control elements. The central housing portion of the device is typically implanted in a suitable subcutaneous region with the septum positioned immediately below the skin of the patient to facilitate access to the reservoir for refilling purposes.

To deliver the infusate from the reservoir, a catheter is usually attached to the outlet port of the central housing to receive the infusate outflow. The distal end of the catheter is implanted within the patient adjacent to the appropriate therapy site (e.g., at a suitable intrathecal location to allow introduction of an infusate directly into the spinal fluid of the patient). Typically, some mechanism is employed to anchor the catheter so that infusate will continue to be delivered to the appropriate site such as sutures and/or anchoring structures.

Similar anchoring is also used in spinal cord stimulation (SCS) systems. In SCS systems, a pulse generator is typically implanted within a subcutaneous pocket within the patient. An electrical lead is also implanted within the patient. The proximal end of the electrical lead is electrically coupled (either directly or via one or more extensions) to the pulse generator to receive electrical pulses from the pulse generator. The distal end of the electrical lead is positioned with electrodes of the lead disposed within the epidural space of the patient to deliver the electrical pulses to the spinal neural tissue of the patient. The efficacy of the electrical stimulation in treating chronic pain of the patient depends upon applying the electrical pulses to the appropriate neural tissue. Accordingly, it is desired to retain the stimulation lead at a relatively fixed position over time. For that reason, the electrical lead is anchored so that migration of the electrical lead does not occur.

### SUMMARY

In one embodiment, an implantable anchor for holding a catheter in place within a patient, comprises a body comprising a channel for holding the catheter; a plurality of apertures such that at least one aperture is disposed on each side of the spiral channel; and one or more suture structures to allow a surgeon to suture the anchor to tissue of the patient; wherein when a catheter is disposed within the channel and is threaded through the plurality of apertures, the plurality of apertures limit longitudinal displacement of the catheter in either longitudinal direction relative to the anchor without continuously applying a compressive force to the catheter.

The foregoing has outlined rather broadly certain features and/or technical advantages in order that the detailed description that follows may be better understood. Additional features and/or advantages will be described hereinafter which form the subject of the claims. It should be appreciated by those skilled in the art that the conception and specific embodiment disclosed may be readily utilized as a basis for modifying or designing other structures for carrying out the same purposes. It should also be realized by those skilled in the art that such equivalent constructions do not depart from the spirit and scope of the appended claims. The novel features, both as to organization and method of operation, together with further objects and advantages will be better understood from the following description when considered in connection with the accompanying figures. It is to be expressly understood, however, that each of the figures is provided for the purpose of illustration and description only and is not intended as a definition of the limits of the appended claims.

### BRIEF DESCRIPTION OF THE DRAWINGS

FIG. 1 depicts an implantable anchor for anchoring a drug infusion catheter, stimulation lead, or other catheter according to one representative embodiment.

FIG. 2 depicts an implantable anchor disposed against spinal ligaments of a patient according to one representative embodiment.

FIGS. 3-9 depict respective views of an implantable anchor according to one representative embodiment.

FIG. 10 depicts a conventional neurostimulation system that may utilize an anchor according to a representative embodiment.

FIG. 11 depicts a conventional drug pump system that may utilize an anchor according to a representative embodiment.

FIG. 12 depicts a flowchart for the steps involved in implanting an implantable medical device according to one representative embodiment.

FIGS. 13A-13D, 14A-14D, 15A-15D, and 16A-16D respectively depict lead anchors according to alternative embodiments.

### DETAILED DESCRIPTION

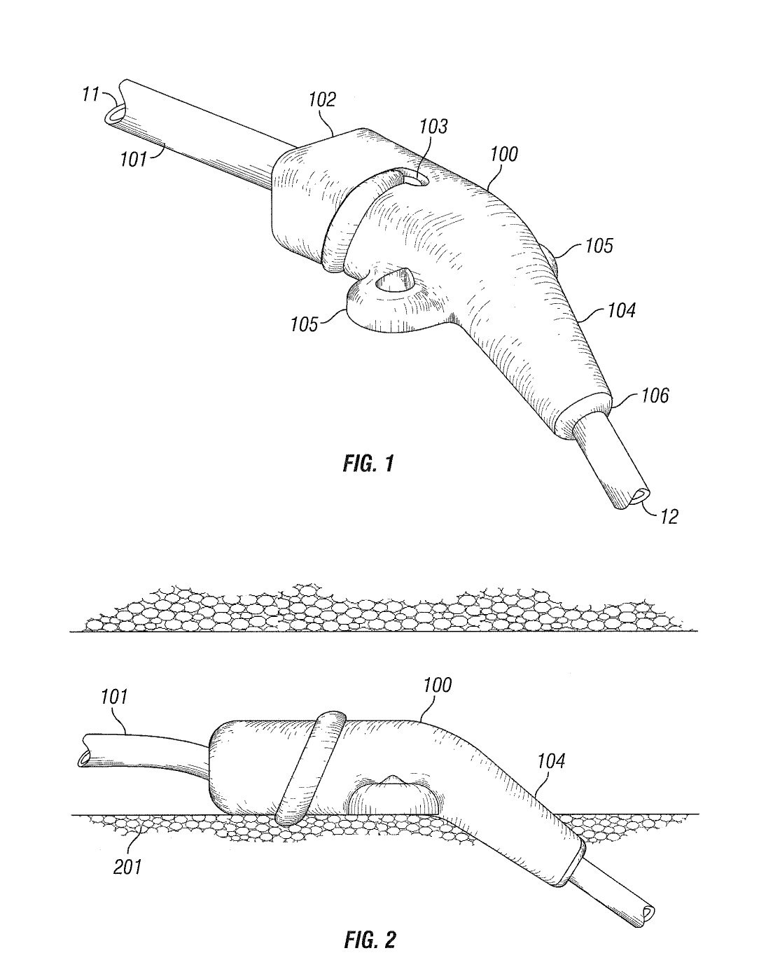

FIG. 1 depicts implantable anchor 100 for anchoring a drug infusion catheter, an electrical lead, or other catheter 101 according to one representative embodiment. For the purpose of this application, the term “catheter” is used in a broad manner and should be interpreted to encompass both infusion catheters and stimulation leads. As shown in FIG. 1, anchor 100 is preferably fabricated or manufactured as a single integral body of polymer material. Accordingly, a surgeon need not assemble relatively small components during the implantation procedure according to some representative embodiments.

Anchor 100 can be fabricated using any suitable polymer processing technique such as injection molding. The polymer selected for anchor is preferably adapted for long term implantation. Biocompatibility and biostability are important characteristics for the polymer selection for anchor 100. Also, the polymer preferably possesses a medium to high durometer to maintain the structural characteristics of anchor 100. An example of a suitable polymer for anchor 100 is silicone, although any biostable, biocompatible polymer having a suitable durometer and a suitable coefficient of friction can be employed.

Anchor 100 differs from conventional catheter anchors in that anchor 100 does not rely upon a continuous application of a compressive force to retain catheter 100 in place. Instead, anchor 100 utilizes a tortuous path or conduit around and, preferably, through anchor 100 to hold anchor 100 in place.

An example of suitable path or conduit for the catheter is shown in FIGS. 1-9. As shown in the embodiment of FIG. 1, catheter 101 is initially threaded into the path through an aperture (not shown) at proximal end 102 of anchor 100. The path or conduit extends through the interior of anchor 100 and emerges at the top of anchor 100 at aperture 103. The path then winds around one side of anchor 100 and re-enters the interior of anchor 100 at another aperture (not shown) on the bottom side of anchor 100. The path extends along the interior of anchor 100 through neck 104 and exits at the distal end of anchor 100 at aperture 106. When catheter 101 is threaded through anchor 100 as shown in FIG. 1, longitudinal forces on catheter 101 cause catheter 101 to hold anchor 100 more tightly thereby preventing displacement of catheter 101. Because anchor 100 does not apply a compressive force to catheter 101 using a suture, there is no technique restriction required by the surgeon to properly employ anchor 100. Specifically, the surgeon need not be concerned with whether suturing is applied too tightly or too loosely.

As shown in the embodiment of FIG. 1, anchor 100 is adapted to allow anchor 100 to be positioned relatively close to spinal ligaments. Specifically, neck portion 104 of anchor 100 is tapered and is angled relative to the body of anchor 100. The distal end 12 of anchor 100 may be positioned within the patient relatively close to spinal ligaments of the vertebral segment where catheter 101 initially enters the epidural space. As shown in FIG. 2, anchor 100 is preferably implanted subcutaneously with the body of anchor 100 resting against or adjacent to spinal ligaments 201 of the patient. Neck portion 104 of anchor 100 extends through the spinal ligaments 201 of the patient with catheter 101 extending through the appropriate vertebral segment (not shown). The angle associated with neck portion 104 tends to prevent catheter 101 from being kinked as the catheter or lead enters the ligament. Specifically, when a catheter or lead is conventionally implanted in a patient disposed in the fetal position, kinking of the lead or catheter can occur when the patient stands and the spine is straightened. Neck portion 104 of anchor mitigates the tendency of the lead to kink in such situations.

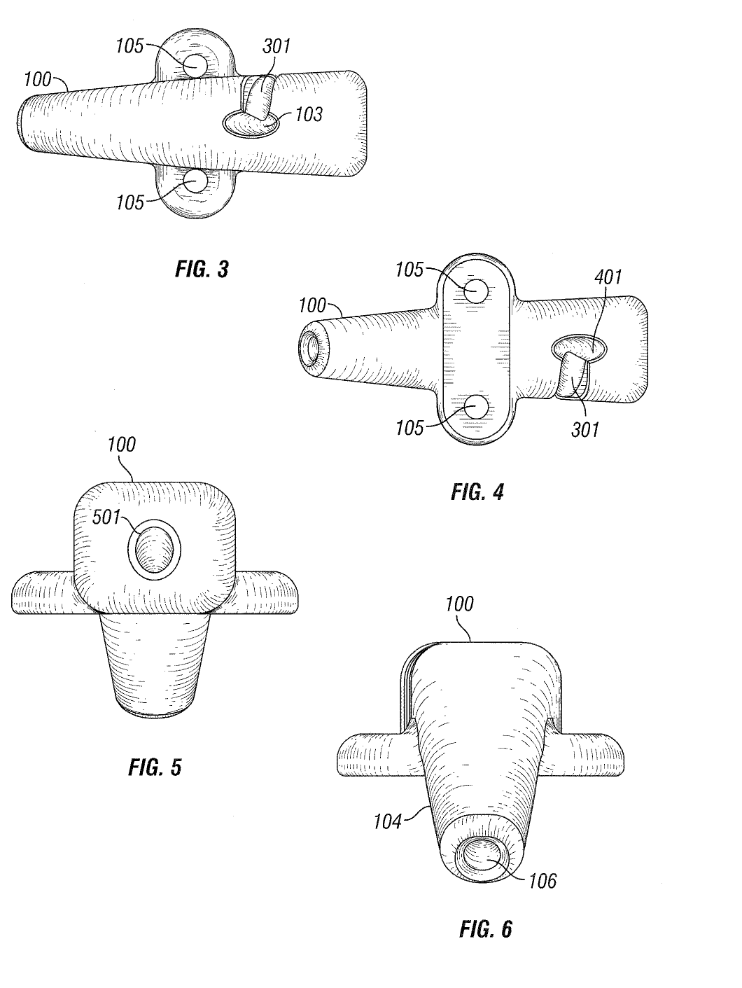

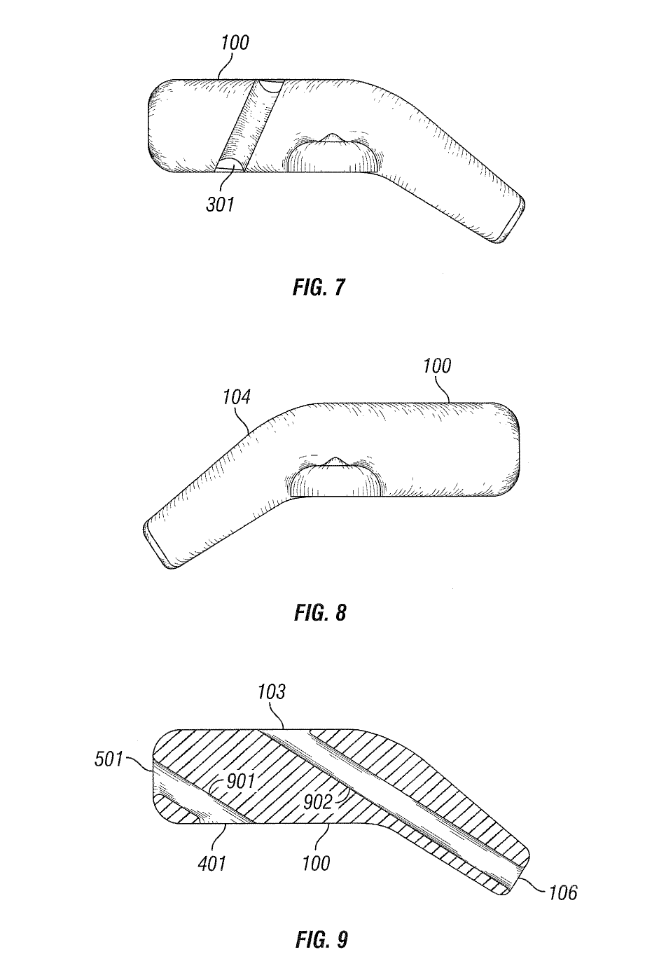

After catheter 101 and anchor 100 are appropriately positioned, anchor 100 can be sutured into place using suture structures 105 disposed on both sides of anchor 100 as shown in FIG. 3. As shown in the top view of FIG. 3, aperture 103 is shown more clearly. Also, path portion 301 is shown extending from aperture 103 and extending around the side of anchor 100. Path portion 301 also extends underneath anchor 100 to aperture 401 as shown in FIG. 4. FIG. 5 depicts a rear view of anchor 100 where catheter 101 is threaded through aperture 501. FIG. 6 depicts a front view of anchor 100 where catheter 101 exits anchor 100 from aperture 106. FIG. 7 depicts a side view of anchor 100 where anchor 100 is adapted to receive catheter 101 along path portion 301 and FIG. 8 depicts the other side of anchor 100. FIG. 9 depicts a sectional view of anchor 100 with interior channels 901 and 902. Interior channel 901 connects between apertures 401 and 501. Interior channel 902 connects between apertures 103 and 106.

Preferably, the entire tortuous path for catheter 101 is adapted such that longitudinal forces on either end of catheter 101 are distributed into the anchor 100 without transferring the force onto the other side of the catheter 101. For example, if a “pulling force” is applied to proximal end 11 of catheter 101 (see FIG. 1), the pulling force is transferred into anchor 100 adjacent to aperture 401 where catheter 101 turns to follow exterior path portion 301. The pulling force is then not transferred to distal end 12 of catheter 101. The sutures attached to suture elements 105 prevent anchor 100 from moving when such a force is applied. Likewise, if a pulling force is applied to distal end of catheter 101, the force is transferred to anchor 100 adjacent to aperture 103 where catheter 101 turns to follow exterior path portion 301 and is not transferred to proximal end 11 of catheter 101.

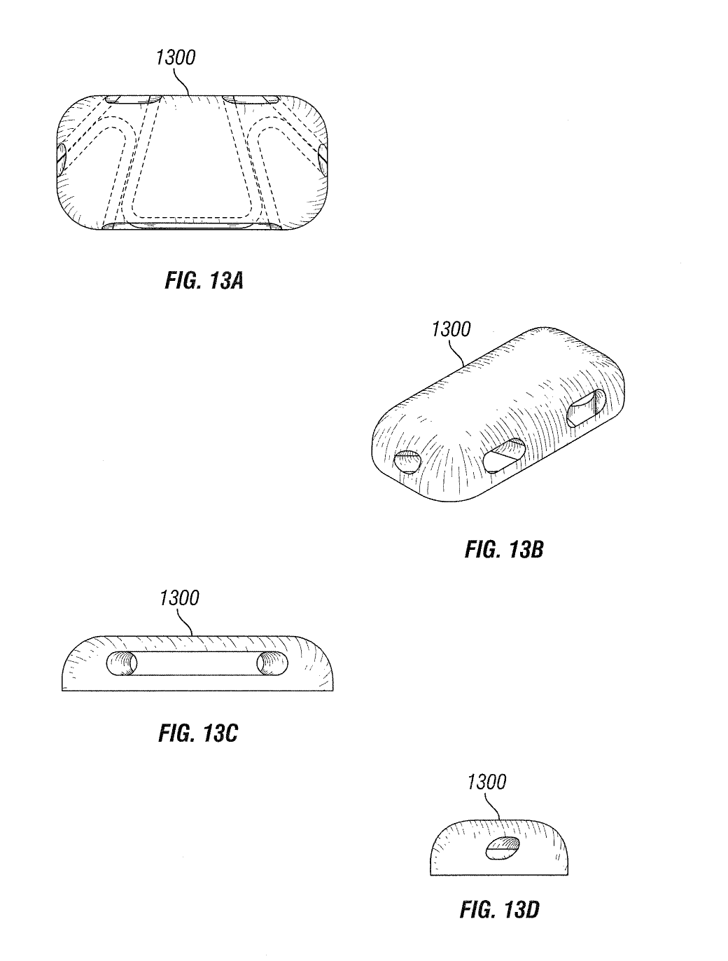

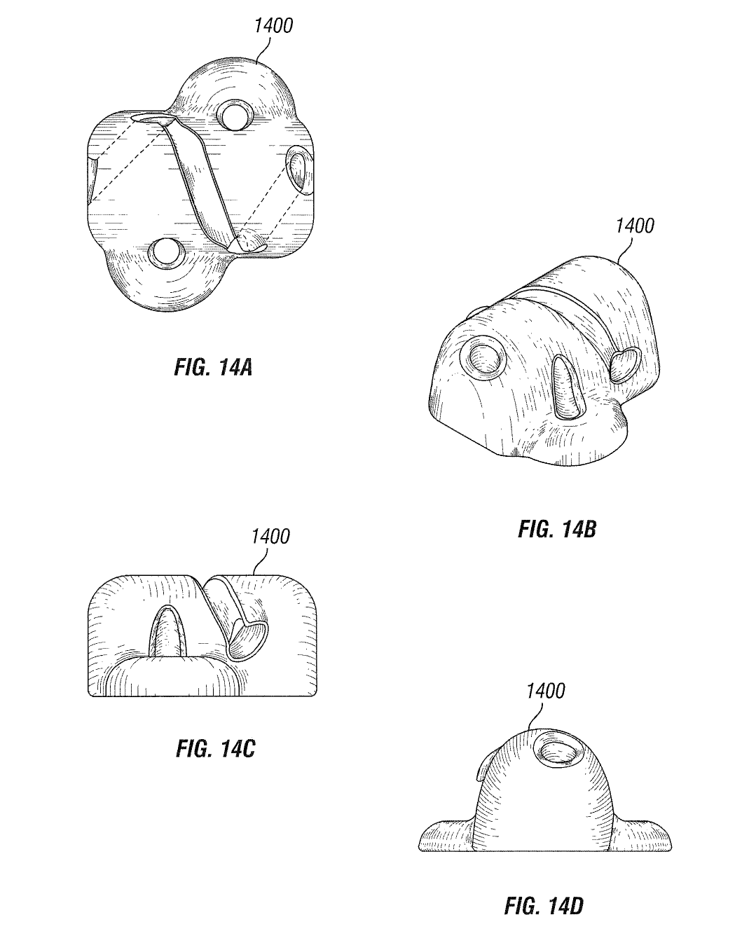

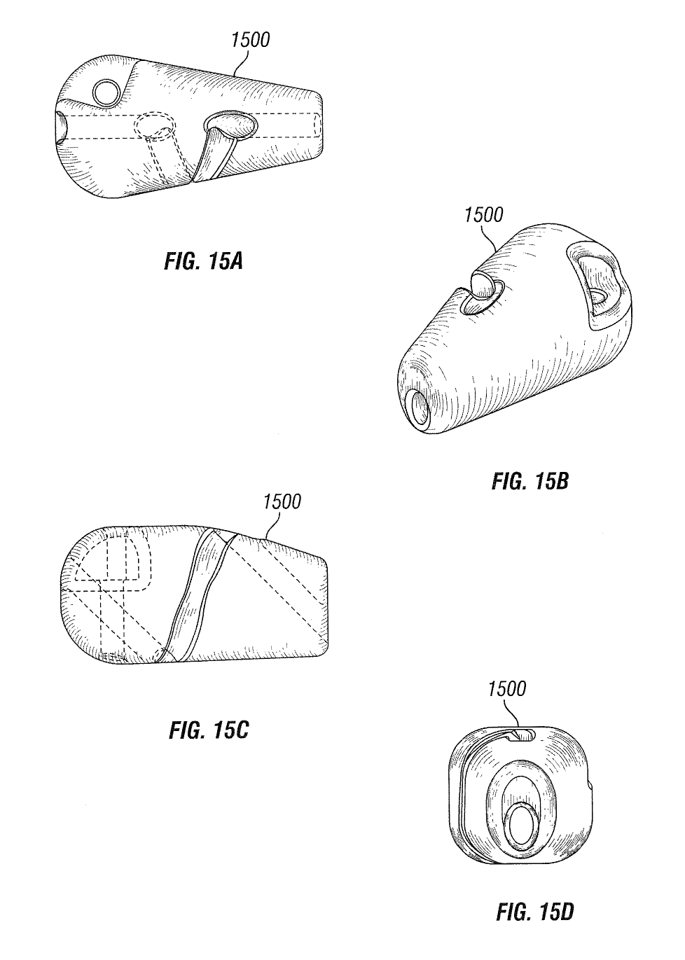

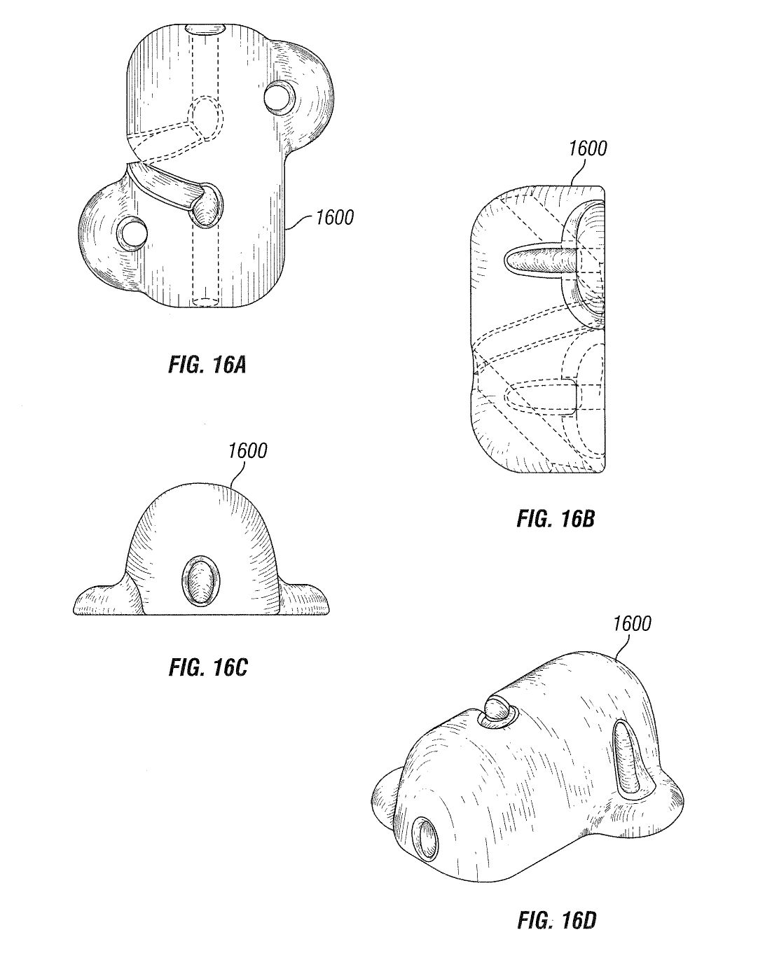

It shall be appreciated that the design shown in FIGS. 1-9 is by way of example. Any suitable path may be employed as long as the path provides sufficient capacity to hold catheter 101 in place. For example, in alternative embodiments, the path could be largely perpendicular to the axis of the anchor. In preferred embodiments, the path for the catheter around or through the anchor comprises two or more structural elements that are disposed to limit longitudinal displacement of the catheter. Utilizing two or more such structural elements enables the transfer of a pulling force from either end of the catheter to the catheter without substantially affecting the other end of the catheter. Additionally, sharper angles for the path at the locations of the structural elements are preferred to apply a greater holding force to the catheter or lead. An issue with conventional compressive anchors is that as the lead or catheter is stretched, its diameter decreases. The reduction in diameter reduces the compressive force on the lead or catheter and, thereby, the lead or catheter may slip. By utilizing suitably adapted structure elements, anchors according to some representative embodiments cause the holding strength of an anchor to increase as the tension on the lead or catheter increases. FIGS. 13A-13D, 14A-14D, 15A-15D, and 16A-16D respectively depict lead anchors 1300, 1400, 1500, and 1600 according to alternative embodiments.

Also, in lieu of or in addition to apertures defined by interior channels, loops, eyelets, grommets, notches, or the like could be defined on the exterior or interior of anchor 100 to facilitate the transfer of pulling forces into the body of the catheter. Preferably, the selected structural elements are disposed on both sides of a spiral channel adapted to accommodate the catheter. Additionally, the catheter can be looped multiple times on the same channel or on different channels according to alternative embodiments.

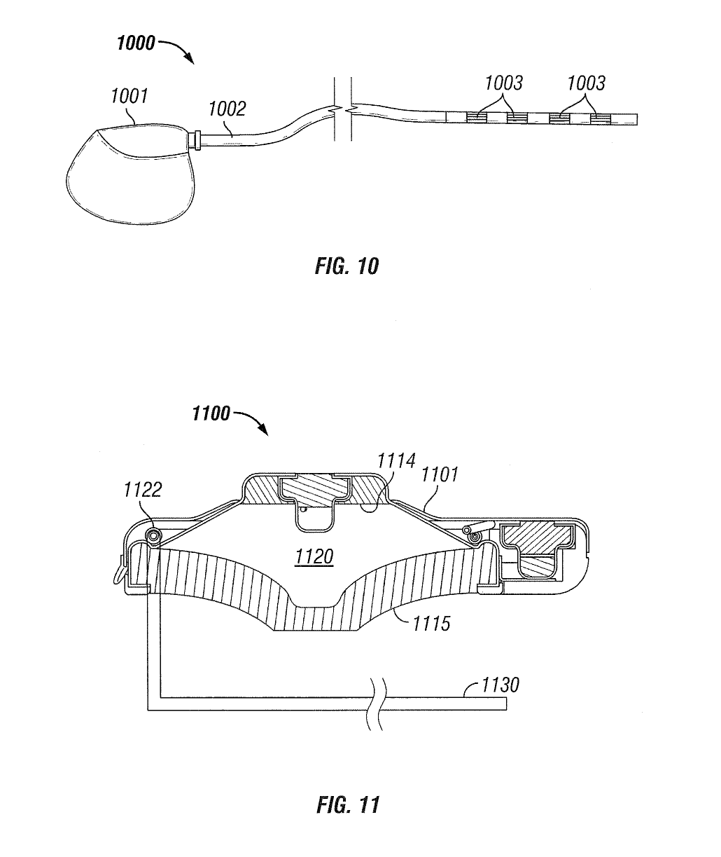

Anchor 100 may be utilized in conjunction with any suitable implantable medical device that comprises an implantable catheter. For example, anchor 100 can be utilized to anchor a stimulation lead of a neurostimulation system as shown in FIG. 10. Neurostimulation system 1000 includes pulse generator 1001 and one or more stimulation leads 1002. An example of a commercially available pulse generator is the EON® product available from Advanced Neuromodulation Systems, Inc. An example of a commercially available stimulation lead is the Axxess® lead available from Advanced Neuromodulation Systems, Inc.

Pulse generator 1001 is typically implemented using a metallic housing that encloses circuitry for generating the electrical pulses for application to neural tissue of the patient. Pulse generator 1001 is usually implanted within a subcutaneous pocket created under the skin by a physician. Lead 1002 is used to conduct the electrical pulses from the implant site of the pulse generator for application to the targeted nerve tissue via electrodes 1003. For example, the distal end of lead 1002 may be positioned within the epidural space of the patient to deliver electrical stimulation to spinal nerves to treat chronic pain of the patient. Anchor 100 may be utilized to ensure that the distal end of lead 1002 remains adjacent to the appropriate nerves associated with the chronic pain of the patient. Also, an “extension” lead (not shown) may be utilized as an intermediate connector if deemed appropriate by the physician. Lead 1002 typically includes a lead body of an insulative polymer material with embedded wire conductors extending through the lead body. Electrodes 1003 on a distal end of the lead body are coupled to the conductors to deliver the electrical pulses to the nerve tissue

In preferred embodiments for SCS applications, catheter 101 is a “body compliant” lead that possesses mechanical characteristics that allow catheter 101 to stretch in response to forces experienced with the patient's body. For example, catheter 101 may be adapted to stretch up to 25% in response to low stretching forces such as 1-2 pounds of force. The ability to exhibit significant elongation in response to such low forces enables the lead to be relatively robust (e.g., does not experience significant conductor breakage). Fabrication techniques and material characteristics for “body compliant” leads are disclosed in greater detail in U.S. Provisional Patent Application Ser. No. 60/788,518, entitled “Lead Body Manufacturing,” filed Mar. 31, 2006, which is incorporated herein by reference.

Alternatively, anchor 100 can be utilized to anchor an infusion catheter of an implantable drug infusion device 1100 as shown in FIG. 11. Implantable infusion drug pump device 1100 may include central housing 1101, reservoir 1120 to hold the infusate, septum 1114 to allow infusate to be introduced into the reservoir, energy source 1115 (e.g., a spring diaphragm) to drive the infusate from the reservoir and through outlet port 1122, and various flow control elements (not shown). Central housing 1101 of the device is often implanted in a suitable subcutaneous region with the septum 1114 positioned immediately below the skin of the patient to facilitate access to reservoir 1120 for refilling purposes. Catheter 1130 is attached to outlet port 1122 of the central housing to receive the infusate outflow. The distal end of the catheter is implanted within the patient adjacent to the appropriate therapy site. An example of a commercially available drug infusion pump device is the AccuRx® product available from Advanced Neuromodulation Systems, Inc.

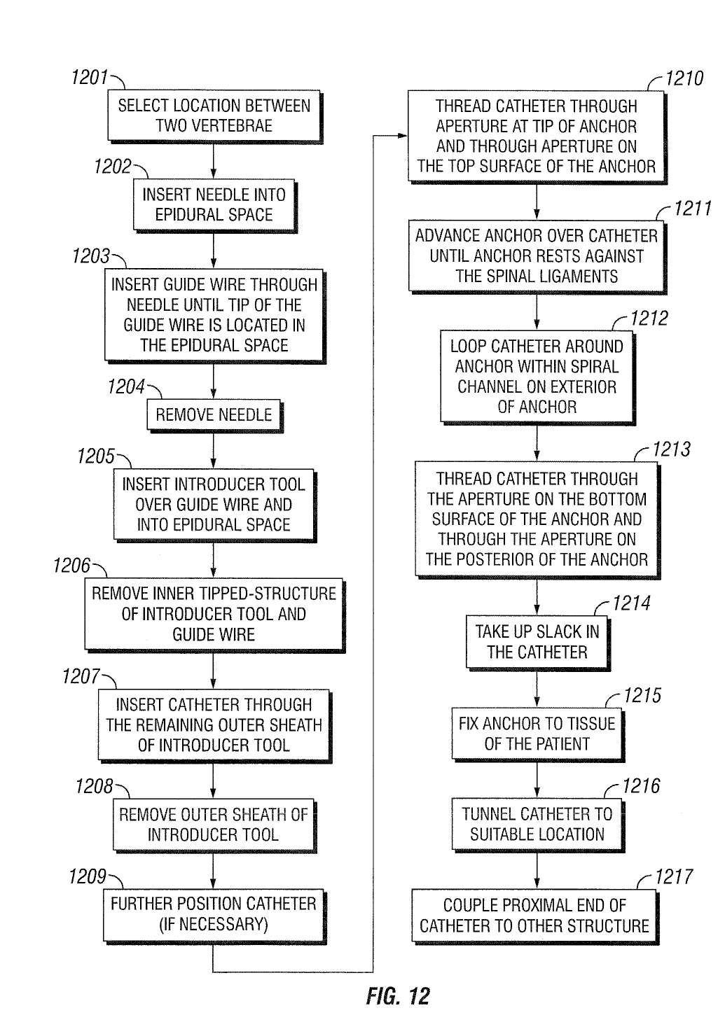

FIG. 12 depicts a flowchart for the steps involved in implanting an implantable medical device according to one representative embodiment.

In step 1201, a location between two vertebrae is selected for the implantation procedure. The specific site may be selected using fluoroscopy. In step 1202, a needle is inserted into the skin, through the subcutaneous tissue and the ligamentum flavum of the spine, and into the patient's epidural space. Entry into the epidural space by needle may be confirmed using standard methods such as the “loss-of-resistance” technique after the stylet or inner portion of the needle is removed.

After removing the stylet from the needle, a guide wire is inserted through the needle into the epidural space (step 1203). Fluoroscopy may be used to verify the proper positioning of the guide wire in the epidural space. A removable stylet may be inserted into a channel extending within the guide wire in order to steer the guide wire to the correct position. Also, the stylet may also provide additional rigidity to the guide wire if desired. Once the tip of the guide wire is in position within the epidural space, the needle is removed (step 1204).

An introducer tool is then inserted (step 1205), preferably at an angle of approximately thirty-five to approximately forty-five degrees, although the exact angle may differ depending on technique and a patient's anatomy, over the guide wire and into the epidural space using the guide wire as a guide. A description of a flexible introducer for catheter implantation is provided in U.S. Patent Publication No. 20050288758, entitled “Methods and apparatuses for implanting and removing an electrical stimulation lead,” which is incorporated herein by reference. The technique of passing the introducer tool over the guide wire helps ensure proper placement of the introducer tool into the epidural space and helps avoid inadvertent passage of the introducer tool into an unsuitable location. Also, the use of a flexible introducer tool enables the introducer tool to advance along flexures in the guide wire and to flex to maneuver around obstructions or physical structures in the body (such as a spinous process, vertebrae, or any other structure in the body) and/or to substantially follow curvatures in the guide wire, rather than displacing portions of the guide wire, which may cause damage to the body.

Once the introducer tool has completely penetrated the ligamentum flavum, an inner tipped-structure of the introducer tool and the guide wire are removed (step 1206) leaving an outer sheath of the introducer tool positioned in the epidural space. The remaining outer sheath provides a channel into the epidural space through which the catheter may be advanced. In step 1207, the catheter is inserted through the outer sheath and positioned at an optimal vertebral level, using fluoroscopy for example, for the desired therapeutic effect. The catheter may be a percutaneous stimulation lead or a laminotomy stimulation lead. Alternatively, the catheter may be a drug infusion catheter and is implanted so that the discharge port of the catheter is preferably disposed within the intrathecal space. The outer sheath of the introducer tool is removed (step 1208) and further positioning of the catheter may occur (step 1209) if necessary.

After the catheter is properly positioned, the catheter is threaded through aperture 106 at the tip of anchor 100 until the catheter exits aperture 103 at the top surface of anchor 100 (step 1210). In step 1211, anchor 100 is advanced over the catheter until anchor 100 contacts the spinal ligaments of the patient (see FIG. 2). The catheter is looped along the exterior portion 301 of the tortuous path of anchor 100 (step 1212). Then the catheter is threaded through aperture 401 on the bottom surface of anchor 100 until the catheter exits aperture 501 on the posterior of anchor 100 (step 1213). In step 1214, any slack in the catheter is taken up by holding anchor 100 in place and pulling the proximal end of the catheter. In step 1215, anchor 100 is fixed in placed by suturing anchor 100 to tissue of the patient using suture structures 105. The catheter is tunneled to a suitable location (step 1216) and coupled to another suitable structure such as a lead extension connector, a pulse generator, or a drug pump (step 1217).

In one alternative embodiment, the material of anchor 100 is a material that is resorbable after implantation within the patient. Specifically, the material of anchor 100 could be resorbed by the patient and replaced with scar tissue over a period of time. Examples of suitable resorbable materials include, but are not limited to, polyglycol acid (PGA), polyactic acid (PLA), polydioxanone (PDO), other polymers and polyesters, and any other material that may be known to those skilled in the art exhibiting the desire functioning and characteristics of ressorbable material. Once the anchor material has been replaced with scar tissue, the tortuous path for the lead remains the same and the lead tends to stay in place when longitudinal forces are applied to the lead. Additionally, the scar tissue would most likely be softer and more pliable than the original material of anchor 100. Accordingly, the patient would experience a greater degree of comfort.

Although some representative embodiments have been discussed in terms of anchoring intrathecal and epidural catheters and leads, anchors can be employed according to alternative embodiments for any suitable location. For example, an anchor possessing a tortuous path could be adapted for peripheral nerve stimulation and gastric pacing applications.

Although representative embodiments and advantages have been described in detail, it should be understood that various changes, substitutions and alterations can be made herein without departing from the spirit and scope of the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure that processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1-30. (canceled)

31-39. (canceled)

40. An apparatus for anchoring a neurostimulation lead in a patient, comprising:
a housing comprising a first portion for anchoring the housing proximate to fascia or other superficial soft tissue and a second portion extending from the first portion for providing strain relief;
a first aperture for receiving the neurostimulation lead at a proximal end of the first portion;
a second aperture for directing the stimulation lead toward an epidural space of the patient;
an internal path through the first and second portions and extending between the first and the second apertures;
wherein the first portion is adapted to be disposed, upon implantation, in planar orientation in subcutaneous tissue of the patient;
wherein the second portion is angled relative to the first portion and, upon implantation, provides an extension from the first portion toward the ligamentum flavum of the patient.

41. The apparatus of claim 40 wherein the first portion extends through ligamentum flavum and into the epidural space of the patient.
